(19) United States
(12) Patent Application Publication
(10) Pub. No.: US 2007/0202061 A1
(43) Pub. Date: Aug. 30, 2007

(54) COSMETIC SKINCARE APPLICATIONS EMPLOYING MINERAL-DERIVED TUBULES FOR CONTROLLED RELEASE

(75) Inventors: Michael D. Riedlinger, Rochester, NY (US); Robert W. Corkery, Stockholm (SE)

Correspondence Address: BASCH & NICKERSON LLP, 1777 PENFIELD ROAD, PENFIELD, NY, 14526, US

(73) Assignee: NaturalNano, Inc., West Henrietta, NY (US)

(21) Appl. No.: 11/554,575

(22) Filed: Oct. 30, 2006

Related U.S. Application Data
(60) Provisional application No. 60/731,488, filed on Oct. 31, 2005.

(51) Int. Cl. A61K 8/49; A61K 8/28

(52) U.S. Cl. 424/59; 424/401

(57) ABSTRACT

Compositions and methods pertaining to the topical treatment of skin are disclosed. The present invention relates to topical compositions for regulating the condition of skin, especially for regulating visible and/or tactile discontinuities in skin associated with skin aging, environmental affects and the like. One embodiment of the present invention relates to improving skin with compositions containing one or more non-volatile, slowly absorbed, liquid or semi-liquid organic substances (active agents) via controlled release of active agents associated with mineral-derived tubules such as halloysite nanotubules. For example, the active agents, in the form of organic substances may include vitamin compounds and glycerin, among others.

This application claims priority from U.S. Provisional Application 60/731,488, for “Cosmetic Skincare Applications for Halloysite Controlled Release,” filed Oct. 31, 2005 by Michael D. Riedlinger, which is hereby incorporated by reference in its entirety.

The present invention relates to topical compositions for regulating the condition of skin, especially for regulating visible and/or tactile discontinuities in skin associated with skin aging, environmental affects and the like. One embodiment of the present invention relates to improving skin with compositions containing one or more non-volatile, slowly absorbed, liquid or semi-liquid organic substances via controlled release of active agents associated with mineral-based tubules such as halloysite nanotubules.

### BACKGROUND AND SUMMARY OF THE INVENTION

Many personal care products currently available to consumers are directed primarily to improving the health and/or physical appearance of the skin. Among these skin care products, some are directed to moisturizing the skin and thereby imparting a resulting healthy appearance to the skin. In addition, these skincare products help to delay, minimize or even reverse skin wrinkling and other histological changes to skin typically associated with aging or environmental damage.

Skin is subjected to various environmental conditions and insults by both extrinsic and intrinsic factors. Extrinsic factors include ultraviolet radiation (e.g., from sun exposure), environmental pollution, wind, heat, low humidity, harsh surfactants, abrasives, and the like. Intrinsic factors include chronological aging and other biochemical changes from within the body. Whether extrinsic or intrinsic, these factors result in visible signs of skin damage and aging, such as wrinkling and other forms of roughness (including increased pore size, flaking and skin lines), and other histological changes. To many people, skin wrinkles are a reminder of the disappearance of youth. As a result, the elimination of wrinkles and halting of similar aging characteristics has become a growing business area in youth-conscious societies. Treatments range from cosmetic creams and moisturizers to various forms of cosmetic surgery.

Extrinsic or intrinsic factors may result in the thinning and general degradation of the skin. For example, as the skin naturally ages, there is a reduction in the cells and blood vessels that supply the skin. There is also a flattening of the dermal-epidermal junction that results in weaker mechanical resistance of this junction. See, for example, Oikarinen, “The Aging of Skin: Chronoaging Versus Photoaging,” Photodermatol. Photoimmunol. Photomed., vol. 7, pp. 3-4, 1990, which is incorporated by reference herein in its entirety.

In accordance with the present disclosure it is believed desirable to provide methods and compositions to more effectively distribute liquid organic substances for maintaining healthy skin. For example, it should be desirable to have a delivery composition in which the components of the composition that act as carriers for the active ingredients have a sustained and controlled effect on the human skin. It may also be desirable to have methods and compositions that provide a controlled release of liquid organic substances. The results, according to the present disclosure, may be obtained as a result of the presence of a mineral phase of the composition essentially constituted of a clay of the kaolinite type, and more particularly of a clay composition having tubule structures, generally referred to herein as mineral-derived tubules.

Controlled release of active agents using nanotubules was reported, for example, by Price, et al., in U.S. Pat. No. 5,651,976. That patent is hereby incorporated by reference in its entirety.

One object of the invention is to provide methods and compositions that have one or more of the attractive facets described above.

The invention provides novel cosmetic applications utilizing mineral-derived tubules. In one aspect, cosmetic applications of the invention include use of mineral-derived microtubes or nanotubes in combination with at least one liquid organic substances. The present disclosure further characterizes exemplary compositions for these applications.

In one aspect, the hollow mineral-derived tubules are selected from one or more clays including halloysite, cylindrite, boulangerite, and imogolite. In one embodiment, the clay microtubules have inner diameters ranging up to about 300 nm. Microtubules may have a pore volume of about 0.23 to about 0.34 ml/g.

In a further aspect of the invention, the liquid organic substances may be selected from liquid organic substances containing glycerin, polyethylene glycol 400, polyethylene glycol 600 and mixtures thereof, and water, wherein the liquid organic substance is present from about 20% to about 55% by volume, and wherein the liquid organic substance may be slowly absorbed into the skin over a period of time,

In a still further aspect, compositions of the invention may include a safe and effective amount of an active agent for regulating skin discontinuities where the active agent comprises a vitamin compound such as a non-vasodilating vitamin B₃ compound, for example niacinamide and a carrier for the active agent, wherein the vitamin B₃ compound is essentially uncomplexed and wherein the vitamin B₃ compound is niacinamide.

In a further aspect of the invention, compositions of the invention include an approximate ratio of active agent to mineral-derived microtubules of between about 1×10⁻⁶:1 to about 10:1. In a further aspect of the invention, the composition includes an approximate ratio of active agent to mineral-derived microtubules (by weight) of 1:1, 2:1, 3:1, 4:1, 5:1.

In a still further aspect of the invention, the compositions of the invention provide an active agent in an extended release profile. For instance, a single application of one of the disclosed compositions may provide effective release of an active agent for about one hour, about two hours, about three hours, about one day, or about seven days.

Mineral-derived tubules used in compositions of the invention that are halloysite may include crude or refined halloysite, or mixtures thereof, and the tubules may be hydrophilic or, with treatment, lipophilic.

The invention contemplates multiple methods of administration of compositions of the invention. For example, compositions of the invention may be applied using cream, ointment, lotion, gel and emulsion. The majority of these products are not miscible with water and likely contain numerous ingredients. For example, a typical composition may contain, but is not limited to containing, about 20 to 40 different ingredients.

In a still further aspect of the invention, the composition provides at least one of these cosmetic compositions loaded or trapped inside, so as to be associated with the mineral-derived tubules. The composition may be loaded or trapped by numerous physical or chemical means including sorption.

Disclosed in embodiments herein is a topical skin composition comprising: mineral-derived tubules; and an active agent, said active agent being at least partially contained within an interior portion of said tubules.

Further disclosed in embodiments herein is a method for the topical treatment of skin, comprising: combining at least one active agent with mineral-derived tubules, wherein said active agent is at least partially contained within the tubules to produce treated tubules; preparing a treatment composition including the treated tubules; and applying the composition to the skin, wherein the active agent is eluted from the tubules over time in order to provide a sustained release of the active agent.

The various aspects and embodiments of the invention are described more fully as set forth herein below and in the associated examples.

### DETAILED DESCRIPTION OF THE INVENTION

Attention is now directed to compositions of mineral-derived microtubules and active agents for cosmetic use, methods of preparing such compositions, and methods of application. The advantages of such compositions may be significant. For instance, they may be safe and effective for use on products meant for eventual consumption. They may be made from natural, environmentally friendly materials. They may provide an extended efficacy due to an extended release profile of an agent in the composition. The may provide beneficial pH properties to skin and hair upon which they are distributed.

Compositions of the invention comprise at least one mineral-derived tubule (e.g., microtubules or nanotubules) and at least one active agent. One such mineral tubule that is naturally occurring is halloysite nanotubules. Halloysite clay is an aluminosilicate with an ideal formula of Al₂Si₂O₅(OH)₄.2H₂O in its hydrated form. Tubules can be formed during weathering of native hydrated clay where the aluminosilicate forms a bilayer structure of distinct alumina and silica layers. The clay consists of subsequent bilayers intercalated by water layers. One of the consequences of this bilayer structure is that as the alumina layer and the silica layer can differ in charge per unit area of cations, and this difference is thought to induce a mean curvature in the otherwise planar sheets of halloysite, forming a scroll-like tube having an open interior region, or lumen. Other elements may substitute into both the octahedral and tetrahedral positions, and these give rise to variations in the physical and chemical properties of halloysites. Other mineral-derived tubules that may be useful in the invention include, for example, but are not limited to those occurring in cylindrite, boulangerite, imogolite, and mixtures thereof.

Halloysite tubules range in length from 100 nm to 40 microns with an average, though dependent on the natural source, around 1.2 microns. Inner diameters range up to 200 nm with an average of approximately 40 nm, with an outer diameter range of about 10-500 nm with an average of about 200 nm. In one embodiment, the cosmetic composition includes halloysite nanoparticles having a mean average cylindrical length of at least about 100 nm to about 40,000 nm, and typically on the order of about 1,200 nm. Inner diameters of untreated nanotubes range up to about 200 nm with an average of approximately 40 nm, while outer diameters range from about 10 nm to 500 nm with an average of about 200 nm. In one embodiment, the cosmetic composition includes halloysite nanoparticles having a mean average outer cylindrical diameter of less than about 500 nm, and preferably on the order of about 200 nm. It may also be possible to characterize the nanotubes using a relationship between certain dimensions, e.g., an aspect ratio (length/diameter). In one embodiment it is believed that halloysite nanotubes may exhibit a length/diameter ratio of between about 0.2 to about 40,000, with an average aspect ratio of about 6.

Native halloysite is a hydrated clay with an intercalated water layer giving a basal spacing of about 10 Å. Subsequent drying of the clay can lead to the dehydrated form of the clay where the intercalated water has been driven off and the basal spacing reduced to about 7 Å. Dehydration is a naturally irreversible process, though researchers have had some success with artificially rehydrating the tubes with a potassium acetate treatment. In the hydrated form small ionic and molecular species can be intercalated in between the bilayers. These small species may include, for example, organic molecules, for example, glycerol and the various active agents as more specifically set forth below. It will be appreciated that the loading of the halloysite may include not only loading of an inter-bilayer space, but also the lumen and/or any inter-bilayer space and/or the outer surface, and/or any microporous or mesoporous space developed in the tubules that are not inter-bilayer, lumenous or exterior surface. As will be further appreciated surface treatment of the tubules may further enhance the loading of the tubules.

In the various embodiments disclosed herein, the halloysite may be used in a crude or refined form. Refined halloysite includes processed halloysite where the nanotubule content has been artificially increased using processing and/or separation technologies, or where the nanotubules have been exfoliated, or both. Those skilled in the art will, with the benefit of this disclosure, recognize that a number of effective methods exist for refining halloysite.

High nanotubule content refined halloysite is particularly useful in the foregoing applications in view of its high surface area. High surface area within the nanotubules permits slow and consistent dissolution or elution of materials (e.g., active agents) loaded within the nanotubule. This permits administration of the agents with extended efficacy and/or extended release profiles.

Many active agents for skin improvement may be selected for use with the invention and inclusion with the mineral-derived microtubules for subsequent elution or dissolution, as discussed and set forth hereinafter. For example, active agents, such as liquid organic substances, may be selected from, but not limited to glycerin, polyethylene glycol 400, polyethylene glycol 600 and other polyethylene glycols with different mean molecular weights. The liquid organic substance is one that is slowly absorbed by the skin surface for about 4 hours to about 18 hours, preferably from about 8 to about 15 hours to achieve, with a usage plan, a skin improvement. As indicated by Chiou et al. in U.S. Pat. No. 6,616,923, issued Sep. 9, 2003 and hereby incorporated by reference in its entirety, such an improvement includes a reduction in the signs of aging including, but not limited to, a reduction in wrinkles, fine lines, and/or age spots. The phrase “skin improvement” also refers to an increase in skin elasticity, softness, smoothness, dewiness, shininess, and/or firmness. “Skin improvement” further refers to moisturizing of skin, treatment of wrinkles, fine lines, age spots and/or the signs of aging, but does not include cleaning of skin. It is further contemplated that the presence of a clay, such as halloysite, may promote oil absorption as an additional benefit of the composition.

As used herein, the phrase “skin cosmetics” refers generally to products used for skin improvement commonly sold in professional cosmetic stores, and the term “treat” includes treating, preventing, ameliorating, or inhibiting a skin condition, including age spots, fine lines, wrinkles, signs of aging, or generally resulting in at least one skin improvement, including an increase in skin elasticity, softness, smoothness, dewiness, shininess, firmness, moisture content and fewer lines, wrinkles and/or age spots.

As used herein, the phrase “slowly absorbed, liquid organic substance” refers to liquid organic substances having high boiling points, such as about 150° C. or higher, that will not evaporate on the surface of skin and can be homogeneously mixed with water or suitable solvent at any ratio. Such substances include, but are not limited to, glycerin, polyethylene glycol 400, polyethylene glycol 600 and other polyethylene glycols with different mean molecular weights. The liquid organic substance is one that may be slowly absorbed by the skin surface for about 4 hours to about 18 hours, preferably from about 8 to about 15 hours.

As used herein, “slowly absorbed” is a qualitative characterization defined as the liquid organic substance being able to physically remain on the skin surface for a long period of time. This is determined by the presence of a layer of the aqueous composition remaining on the skin. For example, since glycerin will not evaporate at 37° C., its disappearance from the skin surface indicates absorption through the skin. An aqueous composition containing 50% propylene glycol, a water miscible, liquid organic substance, disappears from the skin surface in less than two hours, while a composition containing 50% glycerin or polyethylene glycol 400 or 600 remains on the skin for about 4 hours and up to about 18 hours. Compositions useful for carrying out the methods of the invention may consist of one or more water-miscible, liquid organic substances and water, including an aqueous composition of glycerin and water as described in more detail, for example, by Chiou et al. in U.S. Pat. No. 6,616,923, issued Sep. 9, 2003, which is hereby incorporated by reference in its entirety.

Additionally, glycerin can effectively retain water from an aqueous glycerin solution applied on the skin surface. Thus, an aqueous cosmetic product containing glycerin and/or another water-miscible, slowly absorbed liquid organic substance and water applied to the skin surface can serve as a powerful, moisturizing vehicle the effectiveness of which may be further enhanced through the use of mineral-derived tubules as carriers enabling the slow release of such moisturizing agents and compositions.

An appropriate combination of the two active ingredients, a water-miscible, slowly absorbed, liquid organic substance, such as glycerin, and water, stored within the mineral-derived tubules can be used for application to the skin for moisturization and for treatment of dry skin (skin with reduced moisture as compared to normal skin), lines and wrinkles (a line or crease in the skin, such as those caused by sun exposure or old age) and for treatment of dark spots or age spots (skin disorder seen with aging or sun exposure) where there are flat patches of increased pigmentation on the skin.

The aqueous composition used in association with the present invention can contain various amounts of water-miscible liquid organic substance(s) and water. For example, a mixture of glycerin and water, both being endogenous substances in humans, might range from about 10% to about 95% by volume glycerin, and perhaps more preferably about 45% to about 55% by volume glycerin.

Any agents that improve or treat skin when ingested or applied topically may also be easily and inexpensively added to the aqueous composition above described or otherwise associated with the mineral-derived tubules for use as a cosmetic treatment. These agents may include, for example, nutrients such as vitamins (e.g., A, E or C), minerals, amino acids, anti-oxidants, sunscreen agents, or one or more skin peeling compounds (e.g., alpha-hydroxyisobutyric acid). Additionally, one or more preservatives (e.g., isobutylparaben), colorants or fragrances may also be added to the aqueous composition. Such agents may preferably be hypo-allergenic.

The active agent, such as an aqueous composition described above, can be applied to the skin by any appropriate method, which permits the dispersal of a quantity of tubules that have been treated or otherwise have the aqueous composition associated therewith.

Compositions of the invention may include one or more additives. Those skilled in the art will, with the benefit of this disclosure, recognize that a number of alternative additives may be useful in the invention. Additives may include, for example, but are not limited to, one or more colorants, emulsifiers, surfactants, or mixtures thereof. The amount of additive necessary will vary based on the type of additive and the desired effect.

The present invention further contemplates the use of active agents that include vitamin B₃ compounds as described, for example, by Oblong et al. in U.S. Pat. No. 6,238,678, issued May 29, 2001, which is also hereby incorporated by reference in its entirety. Such compounds include nicotinic acid esters, including non-vasodilating esters of nicotinic acid, nicotinyl amino acids, nicotinyl alcohol esters of carboxylic acids, nicotinic acid N-oxide and niacinamide N-oxide.

One or more B vitamin compounds (e.g., B₃) may be used herein, and may be applied through the use of a mineral-derived tubule as previously described. The present invention also contemplates the use of mineral-derived tubules to permit the mixing or combination of a plurality of active agents that might not otherwise be possible in a single cosmetic or skin-care composition.

The present invention contemplates the use of compositions including a dermatologically acceptable carrier such as a mineral-derived tubule within which the vitamin B₃ compound is incorporated to enable the vitamin B₃ compound and optional other active agents to be delivered to the skin at an appropriate concentration and possibly over an extended period of time. The carrier can thus act as a diluent, dispersant, solvent, or the like for the active agent(s), which ensure that it can be applied to and distributed evenly over the selected target at an appropriate concentration.

The carrier may contain one or more dermatologically acceptable solid, semi-solid or liquid fillers, diluents, solvents, extenders and the like. The carrier preferrably includes a mineral-derived tubule such as halloysite nanotubules and the like, and the carrier may itself provide dermatological benefits. Concentrations of the carrier can vary with the carrier selected and the intended concentrations of the essential and optional components. Suitable carriers include conventional or otherwise known carriers that are dermatologically acceptable. The carrier should also be physically and chemically compatible with the essential components described herein, and should not unduly impair stability, efficacy or other use benefits associated with the compositions of the present invention.

The type of material in which the carrier is delivered in the present invention depends on the type of product form desired for the composition. The topical compositions useful in the subject invention may be made into a wide variety of product forms including, but not limited to, solutions, lotions, creams, gels, sticks and other solids, liposomes, sprays (including aerosols), emulsions, ointments, pastes, mousses and cosmetics (e.g., solid, semi-solid, or liquid make-up, including foundations, eye-makeup, pigmented (colored) or non-pigmented lip treatments, e.g., lipsticks, and the like).

The topical compositions may further comprise an emulsion such as oil-in-water emulsions, water-in-oil emulsions, and water-in-silicone emulsions. Emulsions according to the present invention generally contain a solution as described above and a lipid or oil. Lipids and oils may be derived from animals, plants, or petroleum and may be natural or synthetic (i.e., man-made). Preferred emulsions also contain a humectant, such as glycerin. Notably, glycerin is a humectant and feels oily, but not so oily when contained in a porous material. Accordingly, glycerin loaded in mineral-derived tubules will be as good a humectant, or nearly so, but will not have as much “stickiness,” which is a disadvantage of glycerin directly applied to skin.

The emulsion may also contain an anti-foaming agent to minimize foaming upon application to the skin. Anti-foaming agents include high molecular weight silicones and other materials well known in the art for such use.

Other topical compositions include oil-in-water emulsions, having a continuous aqueous phase and a hydrophobic, water-insoluble phase (“oil phase”) dispersed therein. An oil-in-water emulsion may further include a structuring agent to assist in the formation of a liquid crystalline gel network structure.

The oil-in-water emulsions comprise at least one hydrophilic surfactant which can disperse the hydrophobic materials in the water phase (percentages by weight of the topical carrier). The surfactant, at a minimum, must be hydrophilic enough to disperse in water. A wide variety of anionic surfactants may also be useful herein, including those disclosed, for example, in U.S. Pat. No. 3,929,678, to Laughlin et al., issued Dec. 30, 1975, which is incorporated herein by reference in its entirety.

Other anionic materials that may be useful are soaps (i.e. alkali metal salts, e.g., sodium or potassium salts) of fatty acids, typically having from about 8 to about 24 carbon atoms, preferably from about 10 to about 20 carbon atoms. The fatty acids used in making the soaps can be obtained from natural sources such as, for instance, plant or animal-derived glycerides (e.g., palm oil, coconut oil, soybean oil, castor oil, tallow, lard, etc.) The fatty acids can also be synthetically prepared. Soaps are described in more detail in U.S. Pat. No. 4,557,853, which is also hereby incorporated by reference in its entirety. In one embodiment an oil-in-water emulsion may comprise from about 25% to about 98%, water by weight of the topical carrier.

The hydrophobic phase is dispersed in the continuous aqueous phase. The hydrophobic phase may contain water insoluble or partially soluble materials such as are known in the art, including but not limited to, other oils and lipids such as described above in reference to emulsions.

The topical compositions of the subject invention, including but not limited to lotions and creams, may further comprise a dermatologically acceptable emollient. Such compositions preferably contain from about 2% to about 50% of the emollient. As used herein, “emollient” refers to a material useful for the prevention or relief of dryness, as well as for the protection of the skin. A wide variety of suitable emollients are known and may be used herein.

Lotions and creams generally comprise a solution carrier system as described above and one or more emollients. Lotions typically comprise from about 1% to about 20%, of emollient; from about 50% to about 90%, water; and the vitamin B₃ compound. A cream typically comprises from about 5% to about 50%, of emollient; from about 45% to about 85%, water; and the vitamin B₃ compound.

Ointments of the present invention may comprise a simple carrier base of animal or vegetable oils or semi-solid hydrocarbons (oleaginous); absorption ointment bases which absorb water to form emulsions; or water soluble carriers, e.g., a water soluble solution carrier—with the treated mineral-derived tubules dispersed therein.

Compositions useful for cleansing (“cleansers”) may be formulated with a suitable carrier, for example, as described above, and preferably contain, in addition to the vitamin B₃ compound in the above described amounts, from about 1% to about 90%, of a dermatologically acceptable surfactant. The surfactant may be suitably selected from anionic, nonionic, zwitterionic, amphoteric and ampholytic surfactants, as well as mixtures of these surfactants. See U.S. Pat. No. 4,800,197, to Kowcz et al., issued Jan. 24, 1989, which is incorporated herein by reference in its entirety, for exemplary surfactants that may be useful. The cleansing compositions can optionally contain, at their art-established levels, other materials which are conventionally used in cleansing compositions.

As used herein, the term “foundation” refers to a liquid, semi-liquid, semi-solid, or solid skin cosmetic that includes, but is not limited to lotions, creams, gels, pastes, cakes, and the like. Typically the foundation is used over a large area of the skin, such as over the face, to provide a particular look. Foundations are typically used to provide an adherent base for color cosmetics such as rouge, blusher, powder and the like, and tend to hide skin imperfections and impart a smooth, even appearance to the skin. Foundations of the present invention include a dermatologically acceptable carrier for the treated mineral-derived tubules and may include conventional ingredients such as oils, colorants, pigments, emollients, fragrances, waxes, stabilizers, and the like.

In accordance with the various compositions set forth, it will also be appreciated that various means and methods for application of such compositions may be included within the scope of the present invention. For example, a further embodiment could employ mineral-derived tubules loaded with an active agent and applied via a non-woven wipe or the like, so that a quick application of the wipe to the skin delivers a skin benefit agent as described in various embodiments herein.

The topical compositions of the present invention may comprise a wide variety of optional components, provided that such optional components are physically and chemically compatible with the essential components described herein, and do not unduly impair stability, efficacy or other use benefits associated with the compositions of the present invention.

Optional components include aesthetic agents and other active agents. For example, the compositions may include absorbents, abrasives, anticaking agents, antifoaming agents, antimicrobial agents, binders, biological additives, buffering agents, bulking agents, chemical additives, cosmetic biocides, denaturants, cosmetic astringents, drug astringents, external analgesics, film formers, humectants, opacifying agents, fragrances, pigments, colorings, essential oils, skin sensates, emollients, skin soothing agents, skin healing agents, pH adjusters, plasticizers, preservatives, preservative enhancers, propellants, reducing agents, additional skin-conditioning agents, skin penetration enhancing agents, skin protectants, solvents, suspending agents, emulsifiers, thickening agents, solubilizing agents, sunscreens, sunblocks, ultraviolet light absorbers or scattering agents, sunless tanning agents, antioxidants and/or radical scavengers, chelating agents, sequestrants, anti-acne agents, anti-inflammatory agents, anti-androgens, depilation agents, desquamation agents/exfoliants, organic hydroxy acids, vitamins and derivatives thereof, and natural extracts. Such other materials are known and nonexclusive examples of such materials may be found, for example, in Harry's Cosmeticology, 7th Ed., Harry & Wilkinson (Hill Publishers, London 1982); in Pharmaceutical Dosage Forms—Disperse Systems; Lieberman, Rieger & Banker, Vols. 1 (1988) & 2 (1989); Marcel Decker, Inc.; in The Chemistry and Manufacture of Cosmetics, 2nd. Ed., deNavarre (Van Nostrand 1962-1965); in The Handbook of Cosmetic Science and Technology, 1st Ed. Knowlton & Pearce (Elsevier 1993); in “A Consumers' Dictionary of Cosmetic Ingredients” by Ruth Winter (New York, Three Rivers Press, ©2005); and in “Formulary of Cosmetic Preparations” by Michael and Irene Ash (New York, Chemical Publishing, ©1 977).

A safe and effective amount of an anti-inflammatory agent may also be added to the compositions of the subject invention.

Compositions of the present invention may also contain a retinoid. As used herein, “retinoid” includes all natural and/or synthetic analogs of Vitamin A or retinol-like compounds which possess the biological activity of Vitamin A in the skin as well as the geometric isomers and stereoisomers of these compounds. The retinoid may be retinol, retinol esters (e.g., C₂-C₂₂ alkyl esters of retinol, including retinyl palmitate, retinyl acetate, retinyl proprionate), retinal, and/or retinoic acid (including all-trans retinoic acid and/or 13-cis-retinoic acid), more preferably retinoids other than retinoic acid. These compounds are well known in the art and are believed commercially available from a number of sources, e.g., Sigma Chemical Company (St. Louis, Mo.), and Boerhinger Mannheim (Indianapolis, Ind.). Other retinoids which may be useful herein are described in U.S. Pat. No. 4,677,120, issued Jun. 30, 1987 to Parish et al.; U.S. Pat. No. 4,885,311, issued Dec. 5, 1989 to Parish et al.; U.S. Pat. No. 5,049,584, issued Sep. 17, 1991 to Purcell et al.; U.S. Pat. No. 5,124,356, issued Jun. 23, 1992 to Purcell et al.; and U.S. Pat. No. Reissue 34,075, issued Sep. 22, 1992 to Purcell et al.

As used herein, “antimicrobial agent” means a compound capable of destroying microbes, preventing the development of microbes or preventing the pathogenic action of microbes. Antimicrobial agents are useful, for example, in controlling acne. A safe and effective amount of an antimicrobial agent may be added to compositions of the present invention.

Similarly, an “anti-androgen” (a compound capable of correcting androgen-related disorders by interfering with the action of androgens at their target organs) may also be employed. Exposure to ultraviolet light can result in excessive scaling and texture changes of the stratum corneum. Therefore, the compositions of the subject invention preferably contain a sunscreen or sunblock. Suitable sunscreens or sunblocks may be organic or inorganic. A wide variety of conventional sunscreening agents may be suitable for use herein. Also particularly useful in the compositions may be sunscreens such as those disclosed in U.S. Pat. No. 4,937,370 issued to Sabatelli on Jun. 26, 1990, and U.S. Pat. No. 4,999,186 issued to Sabatelli & Spirnak on Mar. 12, 1991, both of which are hereby incorporated by reference in their entirety.

Compositions of the subject invention may also include an anti-oxidant/radical scavenger as an active in addition to the primary active agents. The anti-oxidant/radical scavenger is especially useful for providing protection against UV radiation which can cause increased scaling or texture changes in the stratum corneum and against other environmental agents which can cause skin damage.

A “chelating agent” (an active agent capable of removing a metal ion from a system by forming a complex molecule so that the metal ion cannot readily participate in or catalyze chemical reactions) may also be employed in an embodiment of the invention. The inclusion of a chelating agent is especially useful for providing protection against UV radiation which can contribute to excessive scaling or skin texture changes and against other environmental agents which can cause skin damage.

Compositions of the present invention may further comprise, in one embodiment, an organic hydroxy acid and/or a desquamation agent added to the compositions. In another possible embodiment, the compositions of the present invention may include a safe and effective amount of a depilation agent. The compositions of the present invention may also comprise a skin lightening agent.

The compositions of the present invention may further comprise a humectant, moisturizing agent or other skin conditioning agent. A variety of these materials can be employed including guanidine; glycolic acid and glycolate salts (e.g. ammonium and quaternary alkyl ammonium); lactic acid and lactate salts (e.g. ammonium and quaternary alkyl ammonium); aloe vera in any of its variety of forms (e.g., aloe vera gel); polyhydroxy alcohols such as sorbitol, glycerol, hexanetriol, propylene glycol, butylene glycol, hexylene glycol and the like; polyethylene glycols; sugars and starches; sugar and starch derivatives (e.g., alkoxylated glucose); hyaluronic acid; lactamide monoethanolamine; acetamide monoethanolamine; and mixtures thereof.

The compositions of the present invention may also include an extract obtained by suitable physical and/or chemical isolation from natural sources (e.g., plants, fungi, by-products of microorganisms), including those known in the topical personal care art. Extracts believed suitable are those which enhance the skin appearance benefits of the present invention, and which are used in a safe and effective amount; such extracts include plant and fungal extracts such as extracts of yeast, rice bran, and of the plant Centella Asiatica.

Compounds which are known to stimulate the production of collagen can also be used in the present invention.

Other examples of additional components useful herein include the following: water-soluble vitamins and derivatives thereof (e.g., vitamin C as mentioned above and as described in further detail in the examples below).

Also useful may be aesthetic components such as fragrances, pigments, colorings, essential oils, skin sensates, astringents, skin soothing agents, skin healing agents and the like, nonlimiting examples of these aesthetic components include clove oil, menthol, camphor, eucalyptus oil, eugenol, menthyl lactate, witch hazel distillate, bisabolol, dipotassium glycyrrhizinate and the like.

Preparation of Compositions

The compositions of the present invention are generally prepared by conventional methods such as are known in the art of making topical compositions, with the exception that the active agents are introduced to the composition as an agent associated with a mineral-derived tubule carrier. Such methods typically involve mixing of the ingredients in one or more steps to a relatively uniform state, with or without heating, cooling, application of vacuum, and the like.

Methods for Regulating Skin Condition

The compositions of the present invention may be useful for regulating mammalian skin condition (especially human skin, more especially human facial skin), including visible and/or tactile discontinuities in skin, signs of skin aging, and visible and/or tactile discontinuities in skin associated with skin aging (including fine lines, wrinkles, large pores, surface roughness and other texture discontinuities associated with aged skin). Such regulation includes prophylactic and therapeutic regulation.

Regulating skin condition involves topically applying to the skin a safe and effective amount of a composition of the present invention and the controlled or prolonged release of the active agents described. The amount of the composition which is applied, the frequency of application and the period of use will vary widely depending upon the level of active agents and/or other components of a given composition and the level of regulation desired, e.g., in light of the level of skin aging present in the subject and the rate of further skin aging.

Regulating skin condition may be practiced by applying a composition in the form of a skin lotion, cream, cosmetic, or the like which is intended to be left on the skin for some esthetic, prophylactic, therapeutic or other benefit (i.e., a “leave-on” composition). After applying the composition to the skin, it is preferably left on the skin for a period of at least about 15 minutes, more preferably at least about 30 minutes, even more preferably at least about 1 hour, most preferably for at least several hours, e.g., up to about 12 hours.

The ratio of active agent to mineral-derived microtubules (by weight) in the composition may be varied to provide varying levels of efficacy, release profile, and distribution. The compositions of the invention may include an approximate ratio of active agent to mineral-derived microtubules of between about 1×10⁻⁶:1 to about 10:1. For example, the composition may include an approximate ratio of active agent to mineral-derived microtubules (by weight) of 1:1, 2:1, 3:1, 4:1, or 5:1.

A composition of the invention may provide an active agent in an extended release profile. A composition may provide an active agent in a controlled release profile. For instance, a single application of a composition of the invention may provide effective activity for about one day about two days, about three days about four days, or about five days.

In a further aspect the invention provides a method for imparting active agent properties to an area, an item, or a surface. For example, compositions of the invention may be applied to human skin or hair. For example, a further embodiment could be the delivery of hair colourants from halloysite nanotubes. Moreover, one embodiment could include the loaded tubules being applied in a manner to assure attachment to hairs on the skin, so as to localize their release to the vicinity of the hair. The agent could then spread to cover more skin area or be localized near the hair.

In the skin care trade, it is also known that small molecules referred to as natural moisturization factors or NMF's are also suitable for delivering skin health benefits, both acute and chronic. It is believed that NMF's act as natural moisturizing and/or lubricating agents in the proteinaceous corneocytes or dead skin cells. A further alternative embodiment would be to deliver NMF's to the skin using halloysite and in particular halloysite nanotubes.

Yet a further alternative embodiment would include the delivery of desquamin agents that aid in the removal of unwanted dead skin cells via enzymatic agents, again delivered from halloysite nanotubes. Many of the skin benefit agents that may be delivered act to enhance the function of the stratum corneum, or what is known less formally in the trade as the skin barrier. Accordingly, a further embodiment may include the delivery of active agents that also enhance the penetration of skin benefit agents in a cosmeceutical sense, so that they pass more quickly though the stratum corneum, or skin barrier. Although colorants are suggested above, it is further contemplated that agents applied to the surface of the nanotubules that effect the visual appearance of the halloysite on skin could be used, including for example, dyes, pigments and scattering bodies.

As will be appreciated, the present invention also contemplates the surface treatment of the inner and/or outer surfaces of the tubules to render the tubules more compatible with the active agents and the formulation matrix, including coloidal stabilizing agents. A further embodiment could be the surface modification of the halloysite nanotubes to promote adherence to a substrate, for example, coating in chitosan to render the tubules cationic or with functional groups that attach covalently to skin, hair or nails.

A further embodiment may include the combined use of halloysite as a hair benefit agent that both delivers an active agent such as a moisturizer, but also delivers a further physical benefit such as hair volumizing through its action as a bridge and/or friction agent between hair strands or by its imparting a straightening effect to hair by adhering parallel to the long-fiber axis of the hair, or alternatively by imparting visual effects such as shine/luster, color, and other special effects to hair through coatings applied in or on the mineral-derived tubules.

The practice of one or more aspects of the invention are illustrated in more detail in the following non-limiting examples in which the loading of skincare agents on and from halloysite nanotubules was evaluated. In the evaluation two samples, Vitamin C and E, were chosen as small, low molecular weight model compounds. One is hydrophilic and the other hydrophobic. Thus one is loaded onto hydrophilic, untreated halloysite nanotubules, the other onto hydrophobically modified halloysite nanotubules. The results set forth in accordance with the following examples demonstrate to those skilled in loading porous materials that many small hydrophilic or hydrophobic skin benefit agents can be loaded onto halloysite or other mineral-derived tubules.

The following procedure was used to prepare the halloysite nanotubules (HNT) used in the evaluation. 20 g of HNT's from Imerys were washed and mildly sonicated for about 2 minutes in 100 ml 0.2M oxalic acid to reduce soluble Fe content. The reduction in iron was a safeguard to reduce possible degradation of skin care actives to be loaded on the tubes. The HNT's were then rinsed until the pH returned to near neutral, and then concentrated and dried prior to loading of vitamin C—“washed HNT's.”

### EXAMPLE 1A

### Vitamin C

In the first example, Vitamin C was chosen as the active agent to be loaded. The following process was employed for loading of the Vitamin C:
1. Weighed out 1-5 g of the washed HNT's;
2. Placed these washed HNT's in a conical flask connected to a vacuum pump;
3. Added 100 ml of a Vitamin C-MilliQ water solution and magnetically stirred the solution;
4. Pumped to apply vacuum in the system (trapped air in the nanotubes was released and observed to bubble through the solution.);
5. Stirred the solution under vacuum for 20 min.;
6. Concentrated the HNT's; and
7. Dried in air at 40-50° C. overnight;

### EXAMPLE 1B

### Vitamin C Release

A sample of the dry, Vitamin C loaded HNT's were placed in a small glass vessel, and an amount of solvent (water) was added so that the halloysite was dispersed by magnetic stirring. Aliquots of the solution were then taken after 5 minutes to 4 hours exposure to the solvent, and the absorption spectrum of the aliquot measured soon after being taken. The release of Vitamin C was confirmed by a UV absorption peak near 250 nm, and by comparison with the spectrum of a vitamin C-water solution reference sample.

### EXAMPLE 2A

### Vitamin E

In the second example, Vitamin E was chosen as the active agent to be loaded. The following process was employed for loading of the Vitamin E in to the nanotubules:
1. Dissolved Vitamin E in ethanol (50 ppm);
2. Weighed out 1-5 g of benzalkonium chloride treated HNT's;
3. Placed the weighed sample in a small conical flask;
4. Added Vitamin E in ethanol in a limited quantity to wet the powder, allowing ethanol to evaporate;
5. Repeated several times (until the loading was relatively high); and
6. Dried the sample at room temperature.

### EXAMPLE 2B

### Vitamin E Release

A sample of the dry, Vitamin E loaded HNT's were subsequently placed in a small glass vessel, and an amount of solvent (ethanol) was added so that the halloysite was dispersed by magnetic stirring. Aliquots of the solution were again taken after 5 minutes to 4 hours exposure to the solvent, and the absorption spectrum of the aliquot measured soon after being taken. The release of vitamin E was confirmed using the UV absorption peak near 290 nm, and by comparison with the spectrum of a vitamin E-ethanol solution reference sample.

### EXAMPLE 3

### Glycerin

In a third example, glycerin was chosen as the active agent to be loaded into the HNT's. The following processes were employed for loading of the glycerin:
1. Weighed approximately 4.0 g of HNT's;
2. Added approximately 0.7-0.2 g glycerin or enough to form a viscous liquid, thick paste, dough-like mass or slightly moistened powder;
4. Added ethanol with vigorous mechanical agitation to homogenize the mixture, so that a minimum of ethanol is added to effect an adequate homogenization and to visibly expel trapped air in the HNT's, resulting in a liquid that is free of lumps, free flowing and with glycerin inside the HNT lumens;
5. Allowed ethanol to evaporate yielding a product with glycerin in the lumen of the HNT's.

In a variation of the above example, glycerin was added to just wet the HNT's with no addition of ethanol. The glycerin was mechanically homogenized into the HNT's, and the resulting thick paste was observed to be less sticky than a similar amount of glycerin added directly to the skin, giving the advantage of its humectancy without so much sticky feel.

In another variation, water was added in excess to 2 g of HNT's in a conical flask and a vacuum applied. Fine bubbles were seen to form indicating that trapped air was removed, allowing the water to infiltrate the lumen. It is further believed that water did not enter the lumen of either hydrophilic or hydrophobic HNT's without a vacuum.

Similarly, when vaccum was applied to 2 g of HNT's in excess ethanol, no bubbles were seen to evolve under vacuum, as the ethanol apparently enters the lumen quickly displacing the air. This was observed as indicated above. It is, therefore, believed possible to load various ethanol soluble, skin-benefitting agents without vacuum if ethanol (and similar low surface tension liquids and their mixtures) are used as solvents. Also, the significance is that we can load many skin care agents that are soluble in water (or solvents miscible with water) by applying a vacuum.

### EXAMPLE 4

### NMR Cryoporometry

NMR cryoporometry, a relatively new technique that detects the melting of liquids frozen in pores, was further employed to confirm loading of the tubules. The melting point of the liquid is a function of the pore size for materials with small pores like HNT's. The results obtained show that some liquids froze in the pores of the HNT lumens, thereby providing proof the liquids are inside and not just on and around the tubules. This is important when considering loading the tubes and their subsequent release profiles, since loading is enabled by the solvent entering the pores, and release from the inside rather than outside is believed to be more desirable and/or controllable.

The samples listed in the following Table were prepared by adding the liquids to the HNT powder placed directly in a 5 mm NMR tube. The tubes were centrifuged for 4-12 hours. During measurements, the samples were cooled quickly down below their freezing point and heated again to record their melting transition. Sample 5 was subjected to this procedure twice (denoted as melting ½ in Table 1).

Samples filled with cyclohexane and cyclooctane exhibited a detectable pore liquid transition. No water melting transition was seen for the hydrophilic or hydrophobic samples, therefore water does not appear to load onto the tubes without a vacuum. Similar results are expected for water—to show the same inner pore filling after application of a vacuum. Benzene did not appear to deter the pores of the hydrophobic treated sample.

The pore radius distribution was calculated by using the Gibbs-Thomson equation for cylindrical pores.

Results compilation: Samples 1-5 are hydrophobic surface treated samples, showing the surface treatment is on the inside since the pores are smaller than the untreated, hydrophilic samples. Samples 6-8 are untreated, hydrophilic HNT's.

TABLE

| | | Composition, mg | | Transition | Mean pore | Specific pore |
|---|---|---|---|---|---|---|
| Sample | | clay | liquid | measured | radius¹, nm | volume², cm³g⁻¹ |
| 1 | SMI/H₂O | 12.0 | 62.1 | melting | undetectable | — |
| 2 | SMI/C₆H₆ | 11.4 | 42.0 | | undetectable | — |
| 3 | SMI/C₆H₁₂ | 16.4 | 46.2 | | 31 | 0.17 |
| 4 | SMI/C₈H₁₆ | 10.3 | 29.4 | | 35 | 0.49 |
| 5 | SMI/C₈H₁₆ | 10.8 | 38.9 | melting½ | 36/— | 1.5/0.49(?) |
| 6 | MP/H₂O | 30.3 | 47.5 | melting | undetectable | — |
| 7 | MP/C₈H₁₆ | 33.7 | 40.2 | | 44 | 0.29 |
| 8 | MP/C₈H₁₆ | 35.2 | 28.1 | | 51 | 0.042 |

¹Within an assumption of cylindrical pore shape.
²For pores <=100 nm.

The invention provides multiple methods of administration of compositions in accordance with the various embodiments disclosed. Ideally, compositions may be distributed using means or equipment that is already used by the person applying the compositions in the form of cream, ointment, lotion, gel, emulsion, etc.

It is contemplated that the foregoing embodiments are merely exemplary, and that numerous alternative embodiments incorporating the inventive concepts disclosed herein are within the skill of the ordinary artisan who is provided with the benefit of this disclosure.

What is claimed is:

1. A topical skin composition comprising:
mineral-derived tubules; and
an active agent, said active agent being at least partially contained within an interior portion of said tubules.

2. The composition of claim 1, wherein said active agent comprises a material selected from the group consisting of:
organic substances;
inorganic substances;
water-miscible substances;
liquids;
colloidal substances;
liquid crystals;
vitamins;
vitamin compounds;
amino acids;
polypeptides;
enzymes; and
proteins.

3. The composition of claim 1, wherein the mineral-derived tubules are selected from the group consisting of: halloysite, cylindrite, boulangerite, and imogolite.

4. The composition of claim 1, wherein said composition further comprises a carrier and where said mineral-derived tubules having said active agent therein are interspersed within said carrier for application to skin.

5. The composition of claim 4, wherein said carrier is selected from the group consisting of: solutions, lotions, creams, gels, solids, liposomes, sprays, emulsions, ointments, pastes, mousses, cosmetics and lip treatments.

6. The composition of claim 1, wherein said active agent comprises glycerin.

7. The composition of claim 1, wherein said active agent comprises vitamin B compounds.

8. The composition of claim 7, wherein said active agent comprises a vitamin B₃ compound.

9. The composition of claim 1, wherein said active agent is selected from the group consisting of: vitamin compounds, minerals, amino acids, fatty acids, anti-oxidants, sunscreen agents, skin peeling compounds, preservatives, colorants, pigments, emulsifiers, humectants, emollients, cleansers, surfactants, anti-foaming agents, structuring agents, liquid fillers, foundations, moisturizers, fragrances, waxes, stabilizers, diluents, solvents, extenders, sunblocks and mixtures thereof.

10. The composition of claim 1, wherein said active agent comprises a material selected from the group consisting of: glycerin and polyethylene glycols.

11. The composition of claim 1, wherein said active agent is selected from the group consisting of: absorbents, abrasives, anticaking agents, antifoaming agents, antimicrobial agents, binders, biological additives, buffering agents, bulking agents, chemical additives, cosmetic biocides, denaturants, cosmetic astringents, drug astringents, external analgesics, film formers, humectants, opacifying agents, fragrances, pigments, colorings, essential oils, skin sensates, emollients, skin soothing agents, skin healing agents, pH adjusters, plasticizers, preservatives, preservative enhancers, propellants, reducing agents, additional skin-conditioning agents, skin penetration enhancing agents, skin protectants, solvents, suspending agents, emulsifiers, thickening agents, solubilizing agents, sunscreens, sunblocks, ultraviolet light absorbers or scattering agents, sunless tanning agents, antioxidants and/or radical scavengers, chelating agents, sequestrants, anti-acne agents, anti-inflammatory agents, anti-androgens, depilation agents, desquamation agents/exfoliants, organic hydroxy acids, vitamins, and derivatives thereof.

12. The composition of claim 1, wherein said mineral-derived tubules are surface treated.

13. A method for the topical treatment of skin, comprising:
combining at least one active agent with mineral-derived tubules, wherein said active agent is at least partially contained within the tubules to produce treated tubules;
preparing a treatment composition including the treated tubules; and
applying the composition to the skin, wherein the active agent is eluted from the tubules over time in order to provide a sustained release of the active agent.

14. The method of claim 13, wherein the mineral-derived tubules are selected from the group consisting of: halloysite, cylindrite, boulangerite, and imogolite.

15. The method of claim 13, wherein sustained release of the active agent provides for slow absorption by the skin surface for a period from about 4 hours to about 18 hours.

16. The method according to claim 13, wherein combining at least one active agent with mineral-derived tubules further comprises:
mixing the active agent with a low surface tension liquid to produce a solution; and
exposing the tubules to the solution to permit the solution to wet the tubules and thereby load the active agent therein.

17. The method according to claim 15, wherein said low surface tension liquid is a solvent.

18. The method according to claim 13, wherein combining at least one active agent with mineral-derived tubules further comprises:
mixing the active agent with water to produce a solution;
exposing the tubules to the solution to produce a mixture; and
applying a vacuum to the mixture to cause the solution to wet inner surfaces of the tubules and thereby load the active agent therein.
